(19) United States
(12) Patent Application Publication
(10) Pub. No.: US 2025/0320173 A1
(43) Pub. Date: Oct. 16, 2025

(54) METHODS FOR PURIFYING A SOLVENT

(71) Applicant: Fujifilm Electronic Materials U.S.A., Inc., N. Kingstown, RI (US)

(72) Inventors: Yuan Chen, Chandler, AZ (US); Cole Kareta, Mesa, AZ (US); Peter Dawson, Gilbert, AZ (US); Bryan Hinzie, Gilbert, AZ (US)

(21) Appl. No.: 18/953,231

(22) Filed: Nov. 20, 2024

Related U.S. Application Data
(60) Provisional application No. 63/633,559, filed on Apr. 12, 2024.

(51) Int. Cl. C07C 29/80; B01D 3/14; B01D 3/36; B01D 36/00; B01D 39/16; C07C 31/10

(52) U.S. Cl. CPC C07C 29/80; B01D 3/143; B01D 3/36; B01D 36/00; B01D 39/1692; C07C 31/10; B01D 2239/1216

(57) ABSTRACT

The present disclosure is directed to methods of purifying a solvent, such as an alcohol including n-propanol. The purified solvent can be used, e.g., as a pre-wet liquid, solution developer, and cleaner in a multistep semiconductor manufacturing process.

### CROSS-REFERENCE TO RELATED APPLICATION

The present application claims priority to U.S. Provisional Application Ser. No. 63/633,559, filed on Apr. 12, 2024, the contents of which are hereby incorporated by reference in their entirety.

### FIELD OF THE DISCLOSURE

The present disclosure relates to methods for purifying a solvent, such as an alcohol including n-propanol. In particular, the present disclosure relates to methods that can be used to obtain a solvent having a high purity, thereby providing low on wafer particle and/or metal counts when used as a wafer pre-wetting solvent.

### BACKGROUND OF THE DISCLOSURE

The semiconductor industry has achieved rapid improvements in integration density of electronic components, which are arisen from continuous reductions in the component size. Ultimately, more of the smaller components are afforded to be integrated into a given area. These improvements are mostly due to the development of new precision and high resolution processing techniques.

During the manufacturing of high resolution integrated circuits (ICs), various processing liquids will come into contact with a bare wafer or a film-coated wafer. For example, the fabrication of a fine metal interconnection typically involves a procedure of coating a base material followed by a pre-wetting liquid before the base material is coated with a composite liquid to form a resist film. These processing liquids, containing proprietary ingredients and various additives, are known to be a source of contamination of IC wafer.

It is believed that even if a trace amount of contaminants is mixed into these chemical liquids, such as a wafer pre-wetting liquid or a developer solution, the resulting circuit patterns may have defects. For example, it is known that the presence of very low levels of metal impurities may interfere with the performance and stability of semiconductor devices. Depending on the kind of metallic contaminants, oxide property can deteriorate, inaccurate patterns can be formed, electrical performance of semiconductor circuits can be impaired, which eventually adversely impact manufacturing yields.

The contamination of impurities, such as metal impurities, fine particles, organic impurities, moisture, and the like, can be inadvertently introduced in a chemical liquid during various stages of the manufacturing of the chemical liquid. Examples include impurities that are presented in a raw material, a by-product generated, or an unreacted reactant remained when the chemical liquid is manufactured, or foreign matters eluded or extracted from the surface of the manufacturing apparatus or from a container equipment, reaction vessels, or the like used in transporting, storing, or reacting. Hence, a reduction or removal of insoluble and soluble contaminants from these chemical liquids used for the production of highly precise and ultra-fine semiconductor electronic circuits is a basic assurance of producing defect-free ICs.

In this respect, it is imperative to significantly improve and to rigorously control the standard and quality of chemical liquid manufacturing processes and systems in order to form high purity chemical liquids, which are indispensable in the fabrication of ultra-fine and immensely precise semiconductor electronic circuits.

### SUMMARY OF THE DISCLOSURE

Accordingly, to form highly precise integrated circuits, the demands for ultra-pure chemical liquids, and the quality improvement and control of these liquids become critical. Specific key parameters targeted for quality improvement and control include: liquid and on-wafer metal reduction, liquid and on-wafer particle count reduction, on-wafer defect reduction, and organic contaminant reduction.

In particular, evolving semiconductor technology requires ultra-high purity solvent, such as an alcohol including n-propanol. For example, in addition to requiring n-propanol with low trace metals and low % water concentrations, the semiconductor industry requires low organic impurities as well. A particularly troublesome organic impurity found in n-propanol is propyl propanoate. This impurity is difficult to remove using conventional distillation conditions due to the presence of propyl propanoate and water co-present as an azeotrope. Such difficulties can arise in other azeotropes including a mixture of an alcohol and an ester.

In view of the above, the present disclosure provides methods of purifying n-propanol, in which the n-propanol is produced with amounts of particles, metallic impurities, organic impurities, and residual moisture within acceptable ranges for semiconductor manufacturing and without the generation or introduction of unknown and unwanted substances. Hence, the occurrence of residue and/or particle defects is suppressed and the yield of semiconductor wafer is improved.

In some embodiments, there are provided methods for purifying n-propanol. Such methods can be performed, for example, by:
distilling feed grade n-propanol in a first distillation column to obtain an intermediate grade n-propanol, wherein the first distillation column is operated at a pressure of less than about 200 Torr;
transferring the intermediate grade n-propanol to a second distillation column, wherein the second distillation column is operated at between about 730 and about 760 Torr; and
distilling the intermediate grade n-propanol in the second distillation column to provide finished goods grade n-propanol.

In some embodiments, the first distillation column is operated at a pressure of less than about 150 Torr. In some embodiments, the first distillation column is operated at a pressure of less than about 100 Torr. In some embodiments, the first distillation column is operated at a pressure of about 50 Torr.

In some embodiments, the second distillation column is operated at a pressure of about 735 to about 745 Torr. In some embodiments, the second distillation column is operated at a pressure of about 740 Torr.

In some embodiments, the first distillation column has an inlet positioned at a location that is from about 80% to about 100% of the height of the first distillation column.

In some embodiments, the second distillation column has an inlet positioned at a location that is from about 0% to about 30% of the height of the second distillation column.

In some embodiments, distilling the feed grade n-propanol in the first distillation column removes one or more impurities having a boiling point lower or higher than a boiling point of n-propanol. In some embodiments, the impurity is propyl propanoate.

In some embodiments, distilling the feed grade n-propanol in the first distillation column is performed in absence of added water or another aqueous solvent. Without wishing to be limited by mechanism or theory, the methods herein allow for distillation without co-feeding of water (or another aqueous solvent), which can facilitate azeotrope distillation but can also increase water content in the finished goods grade n-propanol. The results show surprisingly that the methods herein allow for effective distillation of n-propanol from an azeotrope while avoiding co-feeding of water.

In some embodiments, distilling the intermediate grade n-propanol in the second distillation column removes one or more impurities having a boiling point higher than a boiling point of n-propanol and/or one or more impurities comprising a trace metal, an ionic species, and/or a particle. In some embodiments, the impurity is propyl propanoate. In some embodiments, the impurity is a trace metal (e.g., any described herein), an ionic species, and/or a particle.

In some embodiments, distilling the intermediate grade n-propanol in the second distillation column removes one or more impurities having a boiling point lower than a boiling point of n-propanol and/or one or more impurities comprising a trace metal, an ionic species, and/or a particle. In some embodiments, the impurity is propyl propanoate. In some embodiments, the impurity is a trace metal (e.g., any described herein), an ionic species, and/or a particle.

In some embodiments, the methods described herein further include preheating the feed grade n-propanol to a temperature at least about 20° C. below the boiling point before distilling the n-propanol in the first distillation column, wherein the preheating is performed by a preheater upstream of and in fluid communication with the first distillation column.

In some embodiments, the methods described herein further include passing the feed grade n-propanol through a first filter unit upstream of the first distillation column, wherein the first filter unit comprises a first housing and at least one first filter in the first housing, and the at least one first filter comprises a filtration medium.

In some embodiments, the filtration medium in the at least one first filter comprises a polyolefin, a polyamide, a fluoropolymer, or a copolymer thereof.

In some embodiments, the filtration medium in the at least one first filter comprises polypropylene or polytetrafluoroethylene.

In some embodiments, the filtration medium in the at least one first filter has an average pore size from about 50 nm to about 250 nm.

In some embodiments, at least one first filter is a particle removal filter.

In some embodiments, the methods described herein further include passing the finished goods grade n-propanol through a second filter unit downstream of the second distillation column and optionally through an optional third filter unit downstream of the second filter unit, wherein the second filter unit comprises a second housing and at least one second filter in the second housing, and the at least one second filter comprises a filtration medium, and wherein the third filter unit, if present, comprises a third housing and at least one third filter in the third housing, and the at least one third filter comprises a filtration medium.

In some embodiments, the filtration medium in the at least one second filter comprises a polyolefin, a polyamide, a fluoropolymer, or a copolymer thereof.

In some embodiments, the filtration medium in the at least one second filter comprises nylon, polyolefin, or polytetrafluoroethylene.

In some embodiments, the filtration medium in the at least one second filter has an average pore size from about 2 nm to about 10 nm.

In some embodiments, the at least one second filter and/or the at least one third filter, if present, is a particle removal filter.

In some embodiments, the methods herein further include recirculating the finished goods grade n-propanol exiting the second filter unit and the third filter unit, if present. In some embodiments, the recirculating includes moving the finished goods grade n-propanol exiting the second filter unit to a distilled solvent tank and subsequently passing the finished goods grade n-propanol through the second filter unit and the third filter unit, if present, in which the distilled solvent tank is between and in fluid communication with the second distillation column and the second filter unit.

In some embodiments, the methods described herein further include refluxing the intermediate grade n-propanol exiting the bottom of the second distillation column.

In some embodiments, the methods described further include moving the finished goods grade n-propanol to a product container downstream of and in fluid communication with the second distillation column.

In some embodiments, the finished goods grade n-propanol comprises propyl propanoate at a concentration less than about 200 ppm. In some embodiments, the finished goods grade n-propanol comprises propyl propanoate at a concentration less than about 150 ppm. In some embodiments, the finished goods grade n-propanol comprises propyl propanoate at a concentration less than about 100 ppm.

In some embodiments, the finished goods grade n-propanol comprises less than about 0.05 ppb Al as trace metal, less than about 0.05 ppb Mn as trace metal, less than about 0.05 ppb Zn as trace metal, less than about 0.05 ppb Fe as trace metal, and/or less than about 0.05 ppb Na as trace metal.

In some embodiments, the finished goods grade n-propanol comprises water at a concentration less than about 50 ppm.

Furthermore, the present disclosure provides methods for purifying n-propanol, in which such methods can be extended to other solvents and other azeotropes including such solvents. As such, the present disclosure also provides methods of separating an azeotrope that is a mixture including at least an alcohol and an ester, in which such methods can provide a purified solvent (e.g., a purified alcohol). The purified solvents may be produced with amounts of particles, metallic impurities, organic impurities, and residual moisture within acceptable ranges for semiconductor manufacturing and without the generation or introduction of unknown and unwanted substances.

In some embodiments, there are provided methods for separating an azeotrope. In some embodiments, the azeotrope comprises a mixture of an alcohol and an ester. Such methods can be performed, for example, by:
distilling the azeotrope in a first distillation column to obtain an intermediate grade solvent, wherein the first distillation column is operated at a pressure of less than about 200 Torr;
transferring the intermediate grade solvent to a second distillation column, wherein the second distillation column is operated at between about 730 and about 760 Torr; and
distilling the intermediate grade solvent in the second distillation column to provide finished goods grade solvent.

In some embodiments, the alcohol comprises n-propanol, the ester comprises propyl propanoate, and the finished goods grade solvent comprises finished goods grade n-propanol.

In some embodiments, the finished goods grade solvent comprises finished goods grade alcohol. In some embodiments, the finished goods grade solvent comprises finished goods grade solvent ester.

In some embodiments, the azeotrope comprises a binary mixture comprising the alcohol and the ester. In some embodiments, the azeotrope comprises a ternary mixture comprising the alcohol, the ester, and water. In some embodiments, the azeotrope comprises a quaternary mixture comprising the alcohol, the ester, and two other components (e.g., water or another component).

In some embodiments, the first distillation column is operated at a pressure of less than about 150 Torr. In some embodiments, the first distillation column is operated at a pressure of less than about 100 Torr. In some embodiments, the first distillation column is operated at a pressure of about 50 Torr.

In some embodiments, the second distillation column is operated at a pressure of about 735 to about 745 Torr. In some embodiments, the second distillation column is operated at a pressure of about 740 Torr.

In some embodiments, the first distillation column has an inlet positioned at a location that is from about 80% to about 100% of the height of the first distillation column.

In some embodiments, the second distillation column has an inlet positioned at a location that is from about 0% to about 30% of the height of the second distillation column.

In some embodiments, distilling the azeotrope in the first distillation column removes one or more impurities having a boiling point lower or higher than a boiling point of the solvent. In some embodiments, the solvent is the alcohol or the ester. In some embodiments, the solvent is the alcohol, and the impurity is the ester.

In some embodiments, distilling the azeotrope in the first distillation column is performed in absence of added water or another aqueous solvent. Without wishing to be limited by mechanism or theory, the methods herein allow for distillation without co-feeding of water (or another aqueous solvent), which can facilitate azeotrope distillation but can also increase water content in the finished goods grade solvent.

In some embodiments, distilling the intermediate grade solvent in the second distillation column removes one or more impurities having a boiling point higher than a boiling point of the solvent and/or one or more impurities comprising a trace metal, an ionic species, and/or a particle. In some embodiments, the solvent is the alcohol or the ester. In some embodiments, the solvent is the alcohol, and the impurity is the ester. In some embodiments, the impurity is a trace metal (e.g., any described herein), an ionic species, and/or a particle.

In some embodiments, distilling the intermediate grade solvent in the second distillation column removes one or more impurities having a boiling point lower than a boiling point of the solvent and/or one or more impurities comprising a trace metal, an ionic species, and/or a particle. In some embodiments, the solvent is the alcohol or the ester. In some embodiments, the solvent is the alcohol, and the impurity is the ester. In some embodiments, the impurity is a trace metal (e.g., any described herein), an ionic species, and/or a particle.

In some embodiments, the methods described herein further include preheating the azeotrope to a temperature at least about 20° C. below the boiling point before distilling the azeotrope in the first distillation column, wherein the preheating is performed by a preheater upstream of and in fluid communication with the first distillation column.

In some embodiments, the methods described herein further include passing the azeotrope through a first filter unit upstream of the first distillation column, wherein the first filter unit comprises a first housing and at least one first filter in the first housing, and the at least one first filter comprises a filtration medium.

In some embodiments, the filtration medium in the at least one first filter comprises a polyolefin, a polyamide, a fluoropolymer, or a copolymer thereof.

In some embodiments, the filtration medium in the at least one first filter comprises polypropylene or polytetrafluoroethylene.

In some embodiments, the filtration medium in the at least one first filter has an average pore size from about 50 nm to about 250 nm.

In some embodiments, at least one first filter is a particle removal filter.

In some embodiments, the methods described herein further include passing the finished goods grade solvent through a second filter unit downstream of the second distillation column and optionally through an optional third filter unit downstream of the second filter unit, wherein the second filter unit comprises a second housing and at least one second filter in the second housing, and the at least one second filter comprises a filtration medium, and wherein the third filter unit, if present, comprises a third housing and at least one third filter in the third housing, and the at least one third filter comprises a filtration medium.

In some embodiments, the filtration medium in the at least one second filter comprises a polyolefin, a polyamide, a fluoropolymer, or a copolymer thereof.

In some embodiments, the filtration medium in the at least one second filter comprises nylon, polyolefin, or polytetrafluoroethylene.

In some embodiments, the filtration medium in the at least one second filter has an average pore size from about 2 nm to about 10 nm.

In some embodiments, the at least one second filter and/or the at least one third filter, if present, is a particle removal filter.

In some embodiments, the methods herein further include recirculating the finished goods grade solvent exiting the second filter unit and the third filter unit, if present. In some embodiments, the recirculating includes moving the finished goods grade solvent exiting the second filter unit to a distilled solvent tank and subsequently passing the finished goods grade solvent through the second filter unit and the third filter unit, if present, in which the distilled solvent tank is between and in fluid communication with the second distillation column and the second filter unit.

In some embodiments, the methods described herein further include refluxing the intermediate grade solvent exiting the bottom of the second distillation column.

In some embodiments, the methods described further include moving the finished goods grade solvent to a product container downstream of and in fluid communication with the second distillation column.

In some embodiments, the finished goods grade solvent (e.g., the finished goods grade alcohol) comprises the ester at a concentration less than about 200 ppm. In some embodiments, the finished goods grade solvent (e.g., the finished goods grade alcohol) comprises the ester at a concentration less than about 150 ppm. In some embodiments, the finished goods grade solvent (e.g., the finished goods grade alcohol) comprises the ester at a concentration less than about 100 ppm.

In some embodiments, the finished goods grade solvent (e.g., the finished goods grade alcohol) comprises less than about 0.05 ppb Al as trace metal, less than about 0.05 ppb Mn as trace metal, less than about 0.05 ppb Zn as trace metal, less than about 0.05 ppb Fe as trace metal, and/or less than about 0.05 ppb Na as trace metal.

In some embodiments, the finished goods grade solvent (e.g., the finished goods grade alcohol) comprises water at a concentration less than about 50 ppm.

### BRIEF DESCRIPTION OF THE DRAWINGS

FIG. 1 is a schematic diagram showing an example of a purification system adopted in a method of purifying a solvent (e.g., n-propanol) in accordance with some embodiments of the present disclosure.

FIG. 2 is a schematic diagram showing another example of a purification system adopted in a method of purifying a solvent (e.g., n-propanol) in accordance with some embodiments of the present disclosure.

### DETAILED DESCRIPTION OF THE DISCLOSURE

As defined herein, unless otherwise noted, all percentages expressed should be understood to be percentages by weight to the total weight of a composition. Unless otherwise noted, ambient temperature is defined to be between about 16 and about 27 degrees Celsius (° C.). In the present disclosure, “ppm” means “parts-per-million”, “ppb” means “parts-per-billion”, and “ppt” means “parts-per-trillion”, based on the total weight of a composition.

In general, the disclosure features systems and methods for purifying a solvent (e.g., n-propanol). Solvents (e.g., n-propanol or others) produced by the methods described herein can be used in a wafer processing solution (such as a pre-wetting liquid, a developer solution, a rinsing solution, a cleaning solution, or a stripping solution), or as a solvent for a semiconductor material used in any semiconductor manufacturing process.

The methods of the disclosure achieve optimal solvent purity, by operating a first distillation column at low vacuum (e.g., less than about 200 Torr, less than about 150 Torr, less than about 100 Torr, or about 50 Torr), which forms a co-boiling phase for an azeotrope including a mixture of two or more solvents (e.g., a water-alcohol-ester mixture, such as water-propyl propanoate-propanol) that effectively removes a first solvent from the azeotrope (e.g., removes propyl propanoate from a mixture including water-propyl propanoate-propanol). Subsequently, the intermediate grade solvent (e.g., an intermediate grade alcohol, such as intermediate grade n-propanol) is transferred to a second distillation column, which is operated at close to ambient conditions to further slightly remove the first solvent (e.g., an ester, such as propyl propanoate) at the bottom. Without wishing to be limited by theory, it was found that the purity of finished good solvent (e.g., finished good alcohol, such as finished good n-propanol) is negatively correlated with pressure of the first distillation column. In some embodiments, use of pressure less than 20 Torr can achieve a purity of finished good solvent to be at least about 99.99% (e.g., at least at least about 99.995%, at least about 99.999%, at least about 99.9995%, at least about 99.9999%, about 100%, or 100%). In some embodiments, operating the first distillation column at about 50 Torr and the second distillation column at about 740 Torr resulted in the highest purity solvent (e.g., highest purity alcohol, such as n-propanol), with low water and TM (trace metals) levels that are suitable for high technology node semiconductor applications. The results show surprisingly that the methods herein allow for removal of an impurity having a boiling point higher than the boiling point for the solvent to be purified (e.g., in which n-propanol has a typical boiling point of about 97° C. to about 98° C., such as about 1 atm, while propyl propanoate has a typical boiling point of about 122° C. to about 124° C. at ambient pressures) from the top of the first distillation column. Without wishing to be limited by mechanism or theory, the use of low vacuum (e.g., less than about 200 Torr, less than about 150 Torr, less than about 100 Torr, or about 50 Torr) in the first distillation column allows for removal of an impurity having a boiling point higher than the boiling point for the solvent to be purified (e.g., an alcohol, such as n-propanol).

Typically, a first distillation column is generally configured to remove an impurity having a boiling point that is lower than the desired high purity solvent (e.g., an alcohol, such as n-propanol). Unless otherwise indicated, boiling point is provided at standard atmospheric pressure or ambient pressures.

Furthermore, as described herein, the methods can employ a second distillation column (e.g., operated under ambient conditions, such as about 740 Torr) to further remove impurities remaining in the intermediate grade solvent (e.g., intermediate grade alcohol, such as intermediate grade n-propanol). In some embodiments, the impurity removed by the second column includes one or more of the following: an impurity having a boiling point higher than the boiling point of the solvent to be purified (e.g., boiling point of an alcohol, such as n-propanol), a trace metal, an ionic or non-volatile species, or a combination of any of these. Unless otherwise indicated, the term “solvent” can include one or more solvents (e.g., any described herein).

Prior to being subjected to a purification method of the present disclosure, a solvent (e.g., an alcohol, such as n-propanol) may contain an undesirable amount of contaminants and impurities (such as organic impurities, metal impurities, particles, and moisture). After the solvent is processed by the purification methods of the disclosure, substantial amounts of contaminants and impurities are removed. Pre-processed solvent is also referred to in the present disclosure as “unpurified solvent” or “feed grade solvent,” which may be present in as an “azeotrope.” When the solvent is alcohol, pre-processed alcohol is also referred to in the present disclosure as “unpurified alcohol” or “feed grade alcohol”. When the alcohol is n-propanol, pre-processed n-propanol is also referred to in the present disclosure as “unpurified n-propanol” or “feed grade n-propanol”. The pre-processed solvent can be synthesized in house or commercially available via purchasing from a supplier. A post-processed solvent is also referred to in the present disclosure as a “purified solvent”, “intermediate grade solvent”, or “finished goods grade solvent.” When the solvent is alcohol, a post-processed alcohol is also referred to in the present disclosure as a “purified alcohol”, “intermediate grade alcohol”, or “finished goods grade alcohol.” When the alcohol is n-propanol, post-processed n-propanol is also referred to in the present disclosure as a “purified n-propanol”, “intermediate grade n-propanol”, or “finished goods grade n-propanol.” A “purified solvent”, a “purified alcohol” or a “purified n-propanol” can include impurities limited within predetermined ranges.

In some embodiments, the pre-processed or unpurified solvent (e.g., pre-processed or unpurified azeotrope or alcohol, such as n-propanol) can have a purity of at most about 99.95% (e.g., at most about 99.5%, at most about 99%, at most about 98%, at most about 97%, at most about 96%, or at most about 95%). In some embodiments, the post-processed or purified solvent (e.g., post-processes or purified alcohol, such as n-propanol) obtained from the methods described herein can have a purity of at least about 99.99% (e.g., at least at least about 99.995%, at least about 99.999%, at least about 99.9995%, at least about 99.9999%, or 100%). As mentioned herein, “purity” refers to the weight percentage of the desired solvent (e.g., an alcohol, such as n-propanol) in the total weight of the liquid. Purity can be measured by using a gas chromatography mass spectrometry (GC-MS) device (e.g., a thermal desorption (TD) GC-MS device) or an inductively coupled plasma mass spectrometry (ICP-MS) device.

In general, impurities contained in a pre-processed organic solvent (e.g., an alcohol, such as n-propanol, or as can be present in an azeotrope) can include metallic impurities, particles, and others such as organic impurities (e.g., an ester, such as propyl propanoate) and moisture.

As described herein, metal impurities can be in a form of a solid (e.g., metal simplex, particulate metal-containing compound, and the like). In some embodiments, metal impurities can include a metal selected from the group consisting of alkali metals, alkaline earth metals, main group metals, metalloids, transition metals, post-transition metals, and lanthanide metals. Examples of common metallic impurities include heavy metals such as copper (Cu), iron (Fe), manganese (Mn), aluminum (AI), chromium (Cr), lead (Pb), nickel (Ni), zinc (Zn), and lead (Pb); and alkali or alkaline earth metals such as sodium (Na), potassium (K), magnesium (Mg), and calcium (Ca). Depending on the type of metal, metal impurities can deteriorate oxide integrity, degrade MOS gate stacks, and reduce lifetime of devices. In some embodiments, the content of each metal component in the pre-processed solvent (e.g., pre-processed alcohol, such as n-propanol) ranges from about 0.1 to about 2000 ppt (e.g., from about 200 to about 1000 ppt or from about 500 to about 1000 ppt).

In solvent purified by the methods described herein, the total trace metal content is preferred to be within a predetermined range of from 0 ppt (e.g., at least about 1 ppt, at least about 5 ppt, or at least about 10 ppt) to at most about 200 ppt (e.g., at most about 180 ppt, at most about 160 ppt, at most about 150 ppt, at most about 140 ppt, at most about 120 ppt, at most about 100 ppt, at most about 50 ppt, or at most about 20 ppt) in mass, and the amount of each trace metal (e.g., Al, Mn, Fe, Ni, Cr, Zn, Cu, Pb, K, Mg, Na, or Ca) is preferred to be within a predetermined range of from 0 ppt (e.g., at least about 1 ppt, at least about 2 ppt, or at least about 3 ppt) to at most about 20 ppt (at most about 15 ppt, at most about 10 ppt, at most about 8 ppt, at most about 6 ppt, at most about 5 ppt, at most about 4 ppt, at most about 3 ppt, or at most about 2 ppt) in mass.

In the present disclosure, substances having a size of 0.03 μm or greater are referred to as “particles” or “particulates”. Examples of particles include dust, dirt, organic solid matters, and inorganic solid matters. The particles can also include impurities of colloidal metal atoms. The type of the metal atoms that easily forms colloid is not particularly limited, and can include at least one metal atom selected from the group consisting of Na, K, Ca, Fe, Cu, Mg, Mn, Li, Al, Cr, Ni, Zn, and Pb. In solvent purified by the methods described herein, the total number of the particles having a size of 0.03 μm or more (e.g., 0.05 μm or more) is preferred to be within a predetermined range of at most about 50 (at most about 40, at most about 20, at most about 10, at most about 5, at most about 1, or 0) per 1 ml of the solvent. The number of “particles” in a liquid medium can be counted by a light scattering type in-liquid particle counter and is referred as LPC (liquid particle count).

As described herein, organic impurities are different from the desired solvent and refer to organic matters that are contained in the content of 5000 mass ppm or smaller (4000 ppm or smaller, 3000 ppm or smaller, 2000 ppm or smaller, and 1000 ppm or smaller) with respect to the total mass of the liquid containing the desired solvent and the organic impurities. Organic impurities can be volatile organic compounds that are present in ambient air even inside a clean-room. Some of the organic impurities originate from shipping and storage equipment, while some are presented in a raw material from the start. Other examples of organic impurities include a by-product generated when the solvent is synthesized and/or an unreacted reactant. For example and without limitation, a particularly troublesome organic impurity typically present in feed grade n-propanol is propyl propanoate.

The total content of the organic impurities in a purified solvent is not particularly limited. From a point of improving the manufacturing yield of a semiconductor device, the total content of the organic impurities can be at most about 500 ppm (e.g., at most about 400 ppm, at most about 300 ppm, at most about 200 ppm, at most about 100 ppm, at most about 50 ppm, at most about 20 ppm, at most about 10 ppm) and/or at least about 1 ppm (at least about 10 ppm or at least about 100 ppm) of the purified solvent (e.g., purified alcohol, such as n-propanol). In some embodiments, the purified solvent has a trace amount (e.g., at most about 1 ppm) of any measurable organic impurities. The content of the organic impurities in the solvent described herein can be measured by using a gas chromatography mass spectrometry (GC-MS) device (e.g., a thermal desorption (TD) GC-MS device) or an inductively coupled plasma mass spectrometry (ICP-MS) device.

In some embodiments, the total amount of the moisture or water content can be at most about 500 ppm (e.g., at most about 300 ppm, at most about 200 ppm, at most about 100 ppm, at most about 50 ppm) and/or at least about 5 ppm (e.g., at least about 10 ppm, at least about 50 ppm, at least about 100 ppm, or at least about 150 ppm) of the purified solvent (e.g., purified alcohol, such as n-propanol). In some embodiments, the purified solvent is free of water. The moisture or water content in the solvent described herein can be measured by using a Volumetric or Coulometric Karl Fisher titrator.

As described herein, an azeotrope can include a mixture comprising at least one alcohol and at least one ester. In some embodiments, the azeotrope can include water. The azeotrope can include any useful number of components, such as two components in a binary azeotrope, three components in a tertiary azeotrope, four components in a quaternary azeotrope, etc. The component can be any compound or solvent described herein. In some embodiments, a mixture of compounds can be present, such as isomers which have different structures but have the same number of carbon atoms. In some embodiments, only one isomer can be present, or a plurality of differing isomers can be present.

In some embodiments, the azeotrope comprises a mixture comprising at least one alcohol and at least one ester. Optionally, the azeotrope can further comprise water. In some embodiments, a mixture includes an alcohol and an ester form an azeotrope at 1 atm; and/or the mixture forms an alcohol-ester-water co-boiling phase under vacuum. In some embodiments, the desired solvent comprises at least one alcohol. In some embodiments, the finished goods grade solvent comprises finished goods grade alcohol.

In some embodiments, the azeotrope comprises a mixture comprising n-propanol and propyl propanoate. In some embodiments, the desired solvent comprises n-propanol. In some embodiments, the finished goods grade solvent comprises finished goods grade n-propanol.

As described herein, the methods herein can be used with any solvent or combination of solvents described herein to provide a desired solvent as a finished goods grade solvent. In some embodiments, the method can be used with an azeotrope that is a mixture comprising two or more solvents (e.g., at least one alcohol and at least one ester). In some embodiments, the method can be used with one or more of any solvents described herein. In some embodiments, the finished goods grade solvent comprises any solvent described herein. In some embodiments, the finished goods grade solvent has a purity of at least about 99.99% (e.g., at least at least about 99.995%, at least about 99.999%, at least about 99.9995%, at least about 99.9999%, about 100%, or 100%) for a desired solvent selected from a solvent described herein.

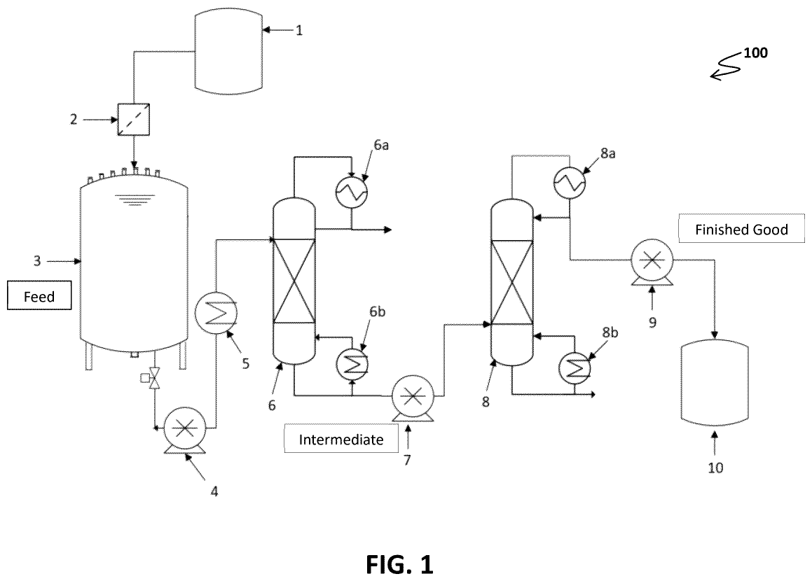

FIG. 1 is a schematic diagram showing a configuration of a purification system according to some embodiments of the present disclosure. As shown in FIG. 1, the purification system 100 includes raw material feed container 1, first filter unit 2, raw material tank 3, pump 4, pre-heater 5, first distillation column 6, condenser 6a, reboiler 6b, pump 7, second distillation column 8, condenser 8a, reboiler 8b, pump 9, and product container 10.

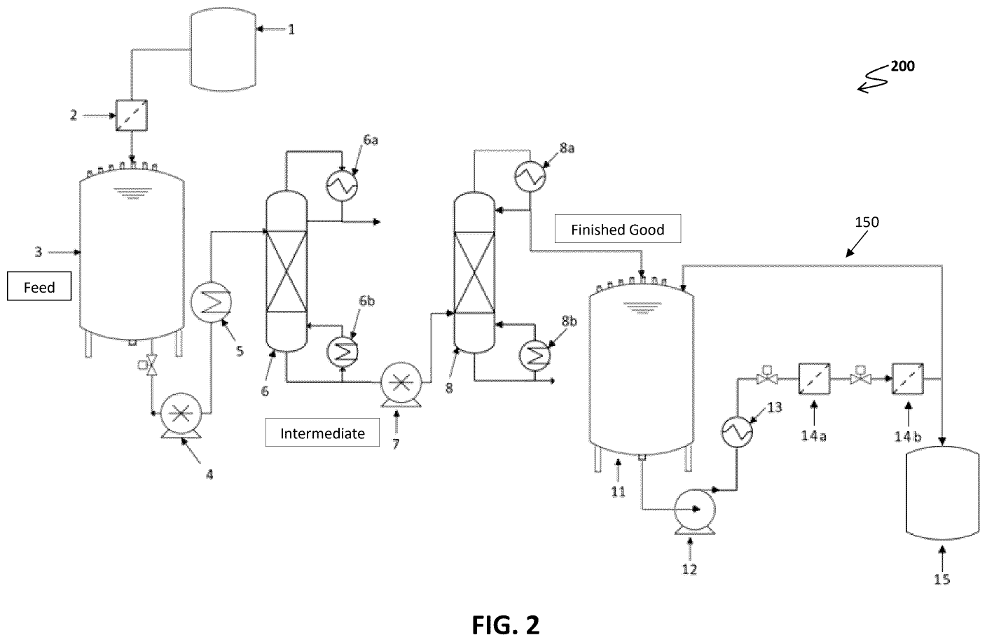

FIG. 2 is a schematic diagram showing a configuration of a purification system according to some embodiments of the present disclosure. As shown in FIG. 2, the purification system 200 includes raw material feed container 1, first filter unit 2, raw material tank 3, pump 4, pre-heater 5, first distillation column 6, condenser 6a, reboiler 6b, pump 7, second distillation column 8, condenser 8a, reboiler 8b, distilled solvent tank 11, pump 12, heat exchanger 13, second filter unit 14a, third filter unit 14b, and product container 15, all of which are in fluid communication with each other (e.g., through one or more pipes or conduits). In purification system 200, distilled solvent tank 11, pump 12, heat exchanger 13, second filter unit 14a, and third filter unit 14b can be optional and can be in fluid connection with one another through an optional recirculation conduit 150 to form a recirculation loop. In general, purification systems 100, 200 can include other components (such as pumps, temperature control units, supply ports, outflow ports, or valves) that may not be shown in FIGS. 1 and 2, respectively. In these figures, like reference numerals refer to like components.

In general, raw material feed container 1 is configured to hold or transport a starting material (e.g., a pre-processed or unpurified azeotrope, or a pre-processed or unpurified alcohol, such as n-propanol). The starting material can be processed by purification system 100 to produce or manufacture a purified solvent (e.g., purified alcohol, including n-propanol) in which the number of unwanted contaminants (e.g., particulates, organic impurities, metallic impurities, and moisture) are limited within predetermined ranges. The type of raw material feed container 1 is not particularly limited as long as it continuously or intermittently supplies the starting material to the other components of purification system 100, 200. In some embodiments, raw material feed container 1 can be a tank, such as a stationary tank or a mobile tank. In some embodiments, raw material feed container 1 can include a material receiving tank, a sensor such as a level gauge (not shown), a pump (not shown), and/or a valve (not shown) for controlling the flow of the starting material (not shown).

Purification system 100, 200 can include at least one (e.g., two or three) pre-distillation filter unit and at least one (e.g., two or three) post-distillation filter unit. In general, the pre-distillation filter unit performs an initial filtration of the starting material (e.g., unpurified solvent, such as unpurified alcohol or unpurified n-propanol, or azeotrope) to remove large particles before distillation, and the post-distillation filter unit performs a filtration after distillation to remove any remaining impurities (e.g., metal or organic impurities) and fine particles to obtain a ultra-high purity solvent (e.g., ultra-high purity alcohol, such as n-propanol). In some embodiments, each of the pre-distillation and post-distillation filter units can include a filter housing and one or more filters (e.g., 1-20 filters) in the filter housing. For example, purification system 100 shown in FIG. 1 includes one pre-distillation filter unit (i.e., first filter unit 2). In another example, purification system 200 shown in FIG. 2 includes one pre-distillation filter unit (i.e., first filter unit 2) and two post-distillation filter units (i.e., second filter unit 14a and third filter unit 14b ). Distillation columns 6 and 8 shown in FIG. 1 or FIG. 2 are generally used to remove the majority of the organic and metal impurities and particles.

In some embodiments, each filter unit in purification system 100, 200 can include a filter housing and one or more (e.g., 2, 3, 4, 5, 6, or 7) filters in the filter housing. Each filter can include a filtration medium made from a suitable material and having an appropriate average pore size. The filters can be arranged in parallel or in series in the filter housing. During use, when two filters are arranged in parallel in a filter housing, solvent to be purified passes these two filters in parallel (i.e., substantially at the same time). On the other hand, when two filters are arranged in series, solvent to be purified passes these two filters sequentially during use. In some embodiments, some filter units can include a plurality of filters in parallel in the filter housing to increase overall flow rate and improve capacity.

For example, purification system 100 shown in FIG. 1 includes one filter unit (i.e., unit 2), which includes a filter housing and one or more filters in the filter housing. In other embodiments, purification system 100 can also include other purification modules in addition to the one filter unit shown in FIG. 1. Purification system 200 shown in FIG. 2 includes three filter units (i.e., units 2, 164, and 14b ), each of which includes a filter housing and one or more filters in the filter housing.

In some embodiments, purification system 100 can include at least one (e.g., two or three) first filter unit 2 between raw material feed container 1 and first distillation column 6 and in fluid communication with container 1 and column 6. First filter unit 2 can include a filter housing and at least one (e.g., 2, 3, 4, or 5) filter in the filter housing.

In some embodiments, when first filter unit 2 includes two or more filters, these filters can be arranged in parallel to improve flow rate and capacity.

In some embodiments, the filters in first filter unit 2 can be a particle removal filter to remove relatively large particles from the organic solvent. In some embodiments, the filters in first filter unit 2 can include a filtration medium having an average pore size of at most about 0.25 μm or 250 nm (e.g., at most about 240 nm, at most about 220 nm, at most about 200 nm, at most about 180 nm, at most about 160 nm, or at most about 150 nm) and/or at least about 0.05 μm or 50 nm (e.g., at least about 60 nm, at least about 70 nm, at least about 80 nm, at least about 90 nm, at least about 100 nm, at least about 110 nm, at least about 120 nm, at least about 130 nm, at least about 140 nm, or at least about 150 nm). Within the above range, it is possible to reliably remove foreign matters such as impurities or aggregates contained in the solvent while suppressing clogging of the filters in first filter unit 2.

Examples of suitable materials of the filtration media in the filters in first filter unit 2 include a fluoropolymer (e.g., polytetrafluoroethylene (PTFE), perfluoroalkoxy alkane polymers (PFA), or a modified polytetrafluoroethylene (MPTFE)), a polyamide such as nylon (e.g., nylon 6 or nylon 66), a polyolefin (including high density and ultrahigh molecular weight resins) such as polyethylene (PE) and polypropylene (PP), or a copolymer thereof. For example, the filtration medium in a particle removal filter can be made of at least one polymer selected from the group consisting of polypropylene (e.g., high density polypropylene), polyethylene (e.g., high density polyethylene (HDPE), or ultra high molecular weight polyethylene (UPE)), nylon, polytetrafluoroethylene, or a perfluoroalkoxy alkane polymer. A filter made of the above materials can effectively remove foreign matters (e.g., those having high polarity) which are likely to cause residue defects and/or particle defects, and to efficiently reduce the content of the metal components in the solvent.

In some embodiments, first filter unit 2 can include two, three, or four filters that are arranged in series, have an average pore size of about 50-200 nm, and include a filtration medium made from polypropylene or polytetrafluoroethylene.

Without wishing to be bound by theory, it is believed that using one or more filters having an average pore size of about 50 nm or less (20 nm or less, 10 nm or less) and including a filtration medium made from polytetrafluoroethylene can significantly reduce the on-wafer metal count and on-wafer particle of the purified solvent described herein. Without wishing to be bound by theory, it is believed that a filter including a filtration medium made from a fluoropolymer (e.g., polytetrafluoroethylene) can produce less on-wafer particles than a filter including a filtration medium made from a polyolefin (e.g., polypropylene).

In some embodiments, purification system 100 can optionally include raw material tank 3 between and in fluid communication with first filter unit 2 and first distillation column 6. The type of raw material tank 3 is not particularly limited as long as it can hold the solvent filtered by first filter unit 2. Without wishing to be bound by theory, it is believed that raw material tank 3 is refilled periodically to provide a sufficient supply of the raw material (e.g., a solvent, such as an alcohol including n-propanol; or a mixture including two or more solvents, such as an azeotrope) for the rest of the purification process and to keep the purification as a continuous process.

In some embodiments, purification system 100 can include a pump 4 and a pre-heater 5 between and in fluid communication with raw material tank 3 and first distillation column 6. During use, pump 4 can deliver solvent (e.g., an alcohol, such as n-propanol; or a mixture of two or more solvents, such as an azeotrope) in raw material tank 3 to pre-heater 5 to be heated to a predetermined temperature and then to first distillation column 6. As used herein, pump 4 can be any suitable pump for transporting or delivering a liquid at operating temperature, such as a metering or diaphragm pump.

In general, pre-heater 5 can be any suitable heating device. Examples of pre-heater include a heat exchanger, an electrical heater, a steam heater, or a mineral oil based heater. In general, pre-heater 5 can heat up the solvent to a desirable temperature. In some embodiments, pre-heater 5 can heat up the solvent to a temperature of from at least about 20° C. (e.g., at least about 19° C., at least about 18° C., at least about 17° C., at least about 16° C., or at most about 15° C.) to at most about 10° C. (e.g., at most about 11° C., at most about 12° C., at most about 13° C., at most about 14° C., or at most about 15° C.) below the boiling point of the desired solvent to be purified. Without wishing to be bound by theory, it is believed that pre-heating the solvent can facilitate removal of low boiling organic impurities from first distillation column 6, allow the purification process to run continuously, and/or improve the efficiency and productivity of the purification process. Further, without wishing to be bound by theory, it is believed that, if the solvent is pre-heated to a temperature that is too high (e.g., within 10° C. below the boiling point of the desired solvent), it can result in temperature overshooting and damaged equipment (e.g., damaged heating element in heat exchanger), reduced product yield (e.g., a certain amount of desired n-propanol can be removed with low boiling or high boiling organic impurities at certain conditions by distillation from the top of first distillation column 6), and an unstable continuous purification process. On the other hand, without wishing to be bound by theory, it is believed that, if the solvent is pre-heated to a temperature that is too low (e.g., more than 20° C. below its boiling point), then the solvent entering first distillation column 6 can be too cold and disrupt the continuing distillation process, which would reduce the overall efficiency of the purification process. In some embodiments, the pre-heater is not needed when under vacuum of 30 Torr or less (20 Torr or less) and the distillation temperature is only 20° C. (or 15° C. or 10° C.) above the ambient temperature.

In some embodiments, purification system 100 includes at least two (e.g., three or four) distillation columns. For example, as shown in FIG. 1, purification system 100 includes first distillation column 6 and second distillation column 8, which are downstream of and in fluid communication with pre-heater 5. In general, distillation columns 6 and 8 can be used to purify the solvent by distillation to remove the majority of the organic and metal impurities and particles. In some embodiments, first distillation column 6 can be used to remove impurities having a boiling point lower than the boiling point of the desired solvent to be purified (e.g., an alcohol, such as n-propanol) or a boiling point higher than the boiling point of the desired solvent to be purified (e.g., an alcohol, such as n-propanol) when a co-boiling phase is formed (e.g., an ester, such as propyl propanoate). In some embodiments, second distillation column 8 can be used to remove impurities having a boiling point higher than the boiling point of the desired solvent to be purified typically, as well as metal impurities, ionic species, and particles that generally cannot be distilled off. Without wishing to be bound by theory, it is believed that switching the order of first distillation column 6 and second distillation column 8 would result in reduced performance, including increased amount of trace metal, increased on-wafer metal count, and/or increased on-wafer particle count.

In some embodiments, first distillation column 6 can include an inlet for receiving the solvent from pre-heater 5 and a first outlet for delivering the solvent to second distillation column 8. In general, the inlet is positioned at a location slightly above the packing material in first distillation column 6, where the separation between the low boiling organic impurities and the desired solvent to be purified occurs. In some embodiments, the inlet can be positioned at a location that is from at least about 80% (e.g., at least about 82%, at least about 84%, at least about 86%, at least about 88%, or at least about 90%) to at most about 100% (e.g., at most about 98%, at most about 96%, at most about 94%, at most about 92%, or at most about 90%) of the height of the first distillation column. Without wishing to be bound by theory, it is believed that placing the inlet at the above location can facilitate the removal of low boiling organic impurities from first distillation column 6, minimize energy required to remove such impurities, and/or increase the efficiency of the purification process. In general, the first outlet can be located at the bottom of the reboiler 6b or the middle of the first distillation column 6. Also without wishing to be bound by theory, it is believed that operation of the first distillation column 6 at vacuum (e.g., 200 Torr or less, 100 Torr or less, 50 Torr or less) can facilitate the formation of co-boiling phase. This results in removal of high boiling organic impurities from first distillation column 6 (e.g., removing an ester from an alcohol, such as removing propyl propanoate from n-propanol) and enables distillation of components from an azeotrope that increases the efficiency of the purification process.

As shown in FIG. 1, first distillation column 6 includes a condenser 6a at the top and a reboiler 6b at the bottom. Condenser 6a can cool or condense the organic impurities exiting a second outlet of first distillation column 6 to form a liquid, which can then be transferred to a waste container. Examples of condenser 6a include water-cooled condensers (such as tube-and-coil, double tube, or tube-and-shell condensers) and air-cooled condensers. Reboiler 6b can provide heat to the solvent to be purified in distillation column 6 to remove impurities having a boiling point lower than the boiling point of the desired solvent to be purified from the top of distillation column 6 and can heat the partially purified solvent to a suitable temperature (e.g., ±2° C. of the boiling point of the desired solvent to be purified) before the solvent is transferred to second distillation column 8 to improve the efficiency and productivity of the purification process. Examples of reboiler 6b include an electrical heater, a steam heater, or a mineral oil based heater.

During operation, upon entering first distillation column 6 through the inlet, the low boiling organic impurities can be distilled off from the top of column 6 through a second outlet for delivering the low boiling organic impurities, cooled by condenser 6a to form a liquid, and transferred to a waste container (not shown in FIG. 1). Because the solvent has been pre-heated to a predetermined temperature, the low boiling organic impurities can be distilled off without travelling down through the packing material in the column to separate them from the solvent to be purified, thereby reducing the energy needed for the distillation and the associated cost. By operating at low pressures or at vacuum (e.g., less than about 200 Torr, less than about 150 Torr, less than about 100 Torr, or about 50 Torr), high boiling impurities can also be distilled off from the top of column 6 through the second outlet mentioned above under certain conditions (e.g., forming a co-boiling phase with water and other chemistry). The solvent to be purified can be collected at the bottom of first distillation column 6 as an intermediate grade solvent and exit column 6 through the first outlet mentioned above at the bottom of the reboiler 6b. The intermediate grade solvent exiting column 6 can be heated by reboiler 6b to a desired temperature and then be delivered to second distillation column 8 through pump 7. Pump 7 can be any high-purity pump with little metal-containing components, thermally rated to the operational temperature, such as a diaphragm pump including PTFE or PEEK (Polyether ether ketone) on a portion or the entire inner surface of the pump.

In some embodiments, second distillation column 8 can include an inlet for receiving the solvent from first distillation column 6 and a first outlet for delivering the distilled solvent to product container 13. In general, the inlet is positioned at a location slightly below the packing material in second distillation column 8. In some embodiments, the inlet can be positioned at a location that is from at least about 0% (e.g., at least about 5%, at least about 10%, at least about 15%, at least about 20%, or at least about 25%) to at most about 30% (e.g., at most about 25%, at most about 20%, at most about 15%, at most about 10%, or at most about 10%) of the height of the second distillation column. Without wishing to be bound by theory, it is believed that placing the inlet at the above location can separate the solvent from high boiling organic impurities (e.g., those having a boiling point higher than the boiling point of the desired solvent to be purified), metal impurities, and particles through distillation and facilitate removal of these impurities from second distillation column 8. In general, the first outlet can be located at the top of condenser 8a of second distillation column 8 and above the packing material in column 8.

As shown in FIG. 1, second distillation column 8 includes a condenser 8a at the top and a reboiler 8b at the bottom. Condenser 8a can cool or condense the solvent to be purified column 6 to form a liquid, which can then be transferred to product container 10 through pump 9. Pump 9 can be any high-purity pump with little metal-containing components, such as a diaphragm pump including PTFE on a portion or the entire inner surface of the pump. Examples of condenser 8a include water-cooled condensers (such as tube-and-coil, double tube, or tube-and-shell condensers) and air- cooled condensers. Reboiler 8b can provide heat to the solvent to be purified so that it can be distilled off from the top of distillation column 8 and impurities having a boiling point higher than the boiling point of the desired solvent can be removed from the bottom of distillation column 8. In some embodiments, reboiler 8b maintains the temperature of the solvent at ±2° C. of the boiling point of the desired solvent to be purified. Examples of reboiler 8b include an electrical heater, a steam heater, or a mineral oil based heater.

During operation, upon entering second distillation column 8 through the inlet, the solvent can be separated from high boiling organic impurities, metal impurities, and particles through distillation and collected from the top of column 8 as a distilled solvent through the first outlet mentioned above at the top of condenser 8a. The high boiling organic impurities, metal impurities, and particles can be collected from the bottom of column 8 through a second outlet for delivering these impurities to a waste container (not shown in FIG. 1).

A purification system can include additional tanks and/or filter units. In some embodiments, purification system 200 can optionally include at least one (e.g., two or three) distilled solvent tank 11 between second distillation column 8 and optional second filter unit 14a and is in fluid communication with column 8 and unit 14a (FIG. 2). In general, distilled solvent tank 11 can be any suitable tank known in the art that can be used to store the distilled solvent (e.g., distilled alcohol, such as distilled n-propanol). In some embodiments, distilled solvent tank 11 can be filled with nitrogen to minimize the moisture and oxidation of the solvent stored in the tank. In some embodiments, during the purification process if the purity level of the distilled solvent exiting second distillation column 8 meets the predetermined requirements (e.g., having a purity of at least about 99.99%, a moisture content of at most about 100 ppm, and/or metal impurities in a total amount of at most about 200 ppt), the solvent can be transferred to product container 15 without passing through filter unit 14a, or filter unit 14b. On the other hand, if the distilled solvent exiting second distillation column 8 does not meet the predetermined requirements, the solvent can first be transferred to distilled solvent tank 11, and then can pass through filter units 164 and/or 14b to remove additional impurities. Similarly, if the purity level of the solvent exiting filter units 164 and/or 14b meets the predetermined requirements, the solvent can be transferred to product container 15. On the other hand, if the purity level of the purified solvent exiting filter units 164 and/or 14b does not meet the predetermined requirements, the solvent can then be transferred back to distilled solvent tank 11 through optional recirculation conduit 150 and be purified again by filter units 164 and/or 14b.

In general, distilled solvent tank 11 can be any suitable vessel for storing a chemical liquid. In some embodiments, distilled solvent tank 11 can have a suitable volume. For example, distilled solvent tank 11 can have a volume of at least about 1000 liters (e.g., at least about 2000 liters, at least about 3000 liters, or at least about 5000 liters) and/or at most about 30,000 liters (e.g., at most about 25,000 liters, at most about 20,000 liters, at most about 15,000 liters, or at most about 10,000 liters).

In some embodiments, when the distilled solvent needs further purification, it can be delivered from distilled solvent tank 11 to second filter unit 14a through pump 12 and heat exchanger 13. Pump 12 can be any pump that can perform recirculation through the tank and filters, such as electromagnetic or centrifuge pumps. In general, heat exchanger 13 can be used to control the temperature of the solvent during the subsequent filtration process.

In some embodiments, purification system 200 can optionally include at least one (e.g., two or three) second filter unit 14a between distilled solvent tank 11 and third filter unit 14b and in fluid communication with tank 11 and unit 14b. In some embodiments, second filter unit 14a can include a filter housing and at least one (e.g., 2, 3, 4, 5, 6, or 7) filters in the filter housing. The filters in second filter unit 14a can be a particle removal filter to remove relatively small particles from the solvent. In some embodiments, the filters in second filter unit 14a can include a filtration medium having an average pore size of at most about 10 nm (e.g., at most about 9 nm, at most about 8 nm, at most about 7 nm, at most about 6 nm, at most about 5 nm, or at most about 4 nm) and/or at least about 2 nm (e.g., at least about 3 nm, at least about 4 nm, at least about 5 nm, at least about 6 nm, at least about 7 nm, or at least about 8 nm). In some embodiments, the average pore size of the filtration medium in the filters in second filter unit 14a can be smaller than the average pore size of the filtration medium in the filters in first filter unit 2. In such embodiments, second filter unit 14a can be used to remove particles smaller than those removed by first filter unit 2.

Examples of suitable materials of the filtration media in the filters in second filter unit 14a include a fluoropolymer (e.g., polytetrafluoroethylene (PTFE), perfluoroalkoxy alkane polymers (PFA), or a modified polytetrafluoroethylene (MPTFE)), a polyamide such as nylon (e.g., nylon 6 or nylon 66), a polyolefin (including high density and ultrahigh molecular weight resins) such as polyethylene (PE) and polypropylene (PP), or a copolymer thereof. For example, the filtration medium in a particle removal filter can be made of at least one polymer selected from the group consisting of polypropylene (e.g., high density polypropylene), polyethylene (e.g., high density polyethylene (HDPE), or ultra high molecular weight polyethylene (UPE)), nylon, polytetrafluoroethylene, or a perfluoroalkoxy alkane polymer.

In some embodiments, second filter unit 14a can include three to seven filters that are arranged in series, have an average pore size of about 5 nm, and include a filtration medium made from nylon.

In some embodiments, purification system 200 can optionally include at least one (e.g., two or three) third filter unit 14b between second filter unit 14a and product container 15 (i.e., downstream of unit 14b ), and in fluid communication with unit 14a and container 15. In some embodiments, third filter unit 14b can include a filter housing and at least one (e.g., 2, 3, 4, or 5) filters in the filter housing. The filters in third filter unit 14b can be a particle removal filter to remove relatively small particles from the solvent. In some embodiments, the filters in third filter unit 14b can include a filtration medium having an average pore size of at most about 10 nm (e.g., at most about 9 nm, at most about 8 nm, at most about 7 nm, at most about 6 nm, at most about 5 nm, or at most about 4 nm) and/or at least about 2 nm (e.g., at least about 3 nm, at least about 4 nm, at least about 5 nm, at least about 6 nm, at least about 7 nm, or at least about 8 nm). In some embodiments, the average pore size or the filtration medium in the filters in third filter unit 14b can be different from the average pore size or the filtration medium in the filters in second filter unit 14a. In such embodiments, third filter unit 14b can be used to remove particles having a different size or nature from those removed by second filter unit 14a. For example, when second filter unit 14a includes filters having a filtration medium made by nylon, third filter unit 14b can include filters having a filtration medium made by PTFE. Without wishing to be bound by theory, it is believed that nylon filters involve a non-sieving mechanism that can remove metal particles, while PTFE filters involve a sieving mechanism that can remove particles based on pore size.

Examples of suitable materials of the filtration media in the filters in third filter unit 14b include a fluoropolymer (e.g., polytetrafluoroethylene (PTFE), perfluoroalkoxy alkane polymers (PFA), or a modified polytetrafluoroethylene (MPTFE)), a polyamide such as nylon (e.g., nylon 6 or nylon 66), a polyolefin (including high density and ultrahigh molecular weight resins) such as polyethylene (PE) and polypropylene (PP), or a copolymer thereof. For example, the filtration medium in a particle removal filter can be made of at least one polymer selected from the group consisting of polypropylene (e.g., high density polypropylene), polyethylene (e.g., high density polyethylene (HDPE), or ultra high molecular weight polyethylene (UPE)), nylon, polytetrafluoroethylene, or a perfluoroalkoxy alkane polymer. A filter made of the above material can effectively remove foreign matters (e.g., those having high polarity) which are likely to cause residue defects and/or particle defects, and to efficiently reduce the content of the metal components in the solvent.

In some embodiments, third filter unit 14b can include two to five filters that are arranged in series, have an average pore size of about 5 nm or less (3 nm or less, 2 nm or less), and are made from polytetrafluoroethylene.

In some embodiments, purification system 200 can optionally include a recirculation conduit 150 to form a recirculation loop (which can include distilled n-solvent tank 11, pump 12, heat exchanger 13, and filter units 164 and 14b ) for recirculating a partially-purified solvent back to distilled solvent tank 11, which can be purified by filter units 164 and/or 14b again. In some embodiments, the partially-purified solvent can be recirculated through the recirculation loop at least two times (e.g., at least three times, at least four times, or at least five times) before the solvent is transferred to product container 15.

In some embodiments, product container 15 can be a mobile storage tank (e.g., a tank on a tanker) or a fixed storage tank. In some embodiments, product container 15 can be a fluoropolymer lined equipment (e.g., the inner surface of which can include a fluoropolymer such as a PTFE). In some embodiments, product container 15 can have a volume of at least about 200 liters (e.g., at least about 300 liters, or at least about 500 liters) and/or at most about 1,500 liters (e.g., at most about 1200 liters, at most about 1000 liters, at most about 900 liters, at most about 800 liters, at most about 700 liters, or at most about 600 liters).

When the number of particles and the amount of impurities detected from the purified solvent at the end of the purification process are controlled within predetermined ranges, an ultra-high purity solvent (e.g., having a purity of at least about 99.99%, a moisture content of at most about 100 ppm, and/or metal impurities in a total amount of at most about 200 ppt) is produced. Subsequently, the ultra-high purity solvent can be transferred to either product container 15 for storage or to a manufacturing process for making a semiconductor article.

In some embodiments, the methods described herein can be either a continuous process or a batch process. When the methods described herein are a continuous process, the solvent can be purified at a relatively high flow rate. For example, the solvent can be purified at a flow rate of at least about 0.2 L/min (e.g., at least about 0.3 L/min, at least about 0.4 L/min, or at least about 0.5 L/min) and/or at most about 1 L/min (e.g., at most about 0.9 L/min, at most about 0.8 L/min, at most about 0.7 L/min, or at most about 0.6 L/min) through purification system 100, 200. In general, the flow rate for purifying a solvent can vary depending on a number of factors, including the temperature, the number of the filters (e.g., those arranged in parallel), the type and number of other equipment used in the purification process.

The present disclosure is illustrated in more detail with reference to the following examples, which are for illustrative purposes and should not be construed as limiting the scope of the present disclosure.

### EXAMPLES

### Example 1

A n-propanol sample was purified in this example. The feed composition included the following: Assay >99.95 wt %, Propyl propanoate <500 ppm, water<300 ppm. Note that water content was not included in the wt % calculation.

Referring to FIG. 1, the feed composition was purified using a system including a first distillation column 6, a pump 7, and a second distillation column 8 that were arranged in the following order: column 6, pump 7, and column 8. In Table 1 below, provided are pressure for the first distillation column (provided as values for “Col 1 Pressure”) and the second distillation column (provided as values for “Col 2 Pressure”). Also provided are concentrations of n-propanol and propyl propanoate for the intermediate grade n-propanol after exiting the first distillation column (provided as values for “Intermediate”) and for the finished goods grade n-propanol after exiting the second distillation column (provided as values for “FG”). Also provided are concentrations of water for the finished goods grade n-propanol after exiting the second distillation column (provided as values for “FG”).

TABLE 1

| | | | | Propyl | | Water |
|---|---|---|---|---|---|---|
| Col 1 | Col 2 | n-propanol (%) | | propanoate (%) | | (ppm) |
| Pressure | Pressure | Inter- | Finished | Inter- | Finished | Finished |
| (Torr) | (Torr) | mediate | Goods | mediate | Goods | Goods |
| 50 | 50 | 99.992 | 99.990 | 0.008 | 0.010 | <50 |
| 740 | 740 | 99.970 | 99.975 | 0.030 | 0.025 | 83 |
| 200 | 740 | 99.978 | 99.980 | 0.022 | 0.020 | <50 |
| 150 | 740 | 99.982 | 99.984 | 0.018 | 0.016 | <50 |
| 110 | 740 | 99.987 | 99.989 | 0.013 | 0.011 | <50 |
| 80 | 740 | 99.989 | 99.991 | 0.011 | 0.009 | <50 |
| 50 | 740 | 99.992 | 99.994 | 0.008 | 0.006 | <50 |

Table 1 presents results obtained while varying column 1 and column 2 pressures employed by the methods described herein. As shown, reducing column 1 pressure while keeping column l pressure close to ambient pressure, results in minimized levels of the organic impurity propyl propanoate, in addition to keeping water concentration at acceptable levels.

Metal content was also evaluated for a non-limiting finished goods grade n-propanol obtained for column 1 pressure being 50 Torr and column 2 pressure being 740 Torr (provided as values for “Distilled” in Table 2), as compared to the raw material (provided as values for “RM” in Table 2).

TABLE 2
Trace Metal Results

| Element | RM (ppb) | Distilled (ppb) |
|---|---|---|
| Li | <0.05 | <0.05 |
| Be | <0.02 | <0.02 |
| Mg | 0.07 | <0.05 |
| Al | 0.12 | <0.05 |
| Ti | <0.1 | <0.1 |
| V | <0.02 | <0.02 |
| Cr | <0.05 | <0.05 |
| Mn | 0.12 | <0.05 |
| Ni | <0.1 | <0.1 |
| Co | <0.05 | <0.05 |
| Cu | <0.05 | <0.05 |
| Zn | 2.4 | <0.05 |
| Ga | <0.02 | <0.02 |
| Ge | <0.05 | <0.05 |
| As | <0.1 | <0.1 |
| Sr | <0.02 | <0.02 |
| Zr | <0.02 | <0.02 |
| Nb | <0.02 | <0.02 |
| Mo | <0.05 | <0.05 |
| Ag | <0.05 | <0.05 |
| Cd | <0.05 | <0.05 |
| Sb | <0.02 | <0.02 |
| Ba | <0.02 | <0.02 |
| Ta | <0.02 | <0.02 |
| W | <0.05 | <0.05 |
| Au | <0.05 | <0.05 |
| Tl | <0.02 | <0.02 |
| Pb | 0.05 | <0.02 |
| Bi | <0.02 | <0.02 |
| Th | <0.05 | <0.05 |
| U | <0.05 | <0.05 |
| Fe | 0.29 | <0.05 |
| Na | 1.7 | <0.05 |
| Ca | 0.19 | <0.05 |
| K | 0.13 | <0.05 |

The data in Table 2 demonstrate that the methods described herein either reduce TM (trace metal) levels (e.g., Al, Mn, Zn) or do not introduce trace metal contamination to the purified n-propanol.

While the invention has been described in detail with reference to certain embodiments thereof, it will be understood that modifications and variations are within the spirit and scope of that which is described and claimed.

What is claimed is:

1. A method for purifying n-propanol, comprising:
distilling feed grade n-propanol in a first distillation column to obtain an intermediate grade n-propanol, wherein the first distillation column is operated at a pressure of less than about 200 Torr;
transferring the intermediate grade n-propanol to a second distillation column, wherein the second distillation column is operated at between about 730 and about 760 Torr; and
distilling the intermediate grade n-propanol in the second distillation column to provide finished goods grade n-propanol.

2. The method of claim 1, wherein the first distillation column is operated at a pressure of less than about 150 Torr.

3. The method of claim 1, wherein the first distillation column is operated at a pressure of less than about 100 Torr.

4. The method of claim 1, wherein the first distillation column is operated at a pressure of about 50 Torr.

5. The method of claim 1, wherein the second distillation column is operated at a pressure of about 735 to about 745 Torr.

6. The method of claim 1, wherein the second distillation column is operated at a pressure of about 740 Torr.

7. The method of claim 1, wherein the first distillation column has an inlet positioned at a location that is from about 80% to about 100% of the height of the first distillation column.

8. The method of claim 1, wherein the second distillation column has an inlet positioned at a location that is from about 0% to about 30% of the height of the second distillation column.

9. The method of claim 1, wherein distilling the feed grade n-propanol in the first distillation column removes one or more impurities having a boiling point lower or higher than a boiling point of n-propanol.

10. The method of claim 1, wherein distilling the feed grade n-propanol in the first distillation column is performed in absence of added water or another aqueous solvent.

11. The method of claim 1, wherein distilling the intermediate grade n-propanol in the second distillation column removes one or more impurities having a boiling point higher than a boiling of n-propanol and/or one or more impurities comprising a trace metal, an ionic species, and/or a particle.

12. The method of claim 1, further comprising preheating the feed grade n-propanol to a temperature at least about 20° C. below the boiling point before distilling the n-propanol in the first distillation column, wherein the preheating is performed by a preheater upstream of and in fluid communication with the first distillation column.

13. The method of claim 1, further comprising passing the feed grade n-propanol through a first filter unit upstream of the first distillation column, wherein the first filter unit comprises a first housing and at least one first filter in the first housing, and the at least one first filter comprises a filtration medium.

14. The method of claim 13, wherein the filtration medium in the at least one first filter comprises a polyolefin, a polyamide, a fluoropolymer, or a copolymer thereof.

15. The method of claim 14, wherein the filtration medium in the at least one first filter comprises polypropylene or polytetrafluoroethylene.

16. The method of claim 13, wherein the filtration medium in the at least one first filter has an average pore size from about 50 nm to about 250 nm.

17. The method of claim 13, wherein the at least one first filter is a particle removal filter.

18. The method of claim 1, further comprising passing the finished goods grade n-propanol through a second filter unit downstream of the second distillation column and optionally through an optional third filter unit downstream of the second filter unit, wherein the second filter unit comprises a second housing and at least one second filter in the second housing, and the at least one second filter comprises a filtration medium, and wherein the third filter unit, if present, comprises a third housing and at least one third filter in the third housing, and the at least one third filter comprises a filtration medium.

19. The method of claim 18, wherein the filtration medium in the at least one second filter comprises a polyolefin, a polyamide, a fluoropolymer, or a copolymer thereof.

20. The method of claim 19, wherein the filtration medium in the at least one second filter comprises nylon, polyolefin, or polytetrafluoroethylene.

21. The method of claim 18, wherein the filtration medium in the at least one second filter has an average pore size from about 2 nm to about 10 nm.

22. The method of claim 18, wherein the at least one second filter and/or the at least one third filter, if present, is a particle removal filter.

23. The method of claim 18, further comprising recirculating the finished goods grade n-propanol exiting the second filter unit and the third filter unit, if present.

24. The method of claim 23, wherein the recirculating comprises moving the finished goods grade n-propanol exiting the second filter unit to a distilled solvent tank and subsequently passing the finished goods grade n-propanol through the second filter unit and the third filter unit, if present, and the distilled solvent tank is between and in fluid communication with the second distillation column and the second filter unit.

25. The method of claim 1, further comprising refluxing the intermediate grade n-propanol exiting the bottom of the second distillation column.

26. The method of claim 1, further comprising moving the finished goods grade n-propanol to a product container downstream of and in fluid communication with the second distillation column.

27. The method of claim 1, wherein the finished goods grade n-propanol comprises propyl propanoate at a concentration less than about 200 ppm.

28. The method of claim 1, wherein the finished goods grade n-propanol comprises propyl propanoate at a concentration less than about 150 ppm.

29. The method of claim 1, wherein the finished goods grade n-propanol comprises propyl propanoate at a concentration less than about 100 ppm.

30. The method of claim 1, wherein the finished goods grade n-propanol comprises less than about 0.05 ppb Al as trace metal.

31. The method of claim 1, wherein the finished goods grade n-propanol comprises less than about 0.05 ppb Mn as trace metal.

32. The method of claim 1, wherein the finished goods grade n-propanol comprises less than about 0.05 ppb Zn as trace metal.

33. The method of claim 1, wherein the finished goods grade n-propanol comprises less than about 0.05 ppb Fe as trace metal.

34. The method of claim 1, wherein the finished goods grade n-propanol comprises less than about 0.05 ppb Na as trace metal.

35. The method of claim 1, wherein the finished goods grade n-propanol comprises water at a concentration less than about 50 ppm.

36. A method for separating an azeotrope comprising a mixture of an alcohol and an ester, the method comprising:
distilling the azeotrope in a first distillation column to obtain an intermediate grade solvent, wherein the first distillation column is operated at a pressure of less than about 200 Torr;
transferring the intermediate grade solvent to a second distillation column, wherein the second distillation column is operated at between about 730 and about 760 Torr; and
distilling the intermediate grade solvent in the second distillation column to provide finished goods grade solvent.

37. The method of claim 36, wherein:
the alcohol comprises n-propanol, the ester comprises propyl propanoate, and the finished goods grade solvent comprises finished goods grade n-propanol.

38. The method of claim 36, wherein the first distillation column is operated at a pressure of less than about 150 Torr.

39. The method of claim 36, wherein the first distillation column is operated at a pressure of less than about 100 Torr.

40. The method of claim 36, wherein the first distillation column is operated at a pressure of about 50 Torr.

41. The method of claim 36, wherein the second distillation column is operated at a pressure of about 735 to about 745 Torr.

42. The method of claim 36, wherein the second distillation column is operated at a pressure of about 740 Torr.

43. The method of claim 36, wherein the first distillation column has an inlet positioned at a location that is from about 80% to about 100% of the height of the first distillation column.

44. The method of claim 36, wherein the second distillation column has an inlet positioned at a location that is from about 0% to about 30% of the height of the second distillation column.

45. The method of claim 36, wherein distilling the azeotrope in the first distillation column removes one or more impurities having a boiling point lower or higher than a boiling point of a solvent, and wherein the solvent comprises the alcohol or the ester.

46. The method of claim 36, wherein distilling the azeotrope in the first distillation column is performed in absence of added water or another aqueous solvent.

47. The method of claim 36, wherein distilling the intermediate grade solvent in the second distillation column removes one or more impurities having a boiling point higher than a boiling of the solvent and/or one or more impurities comprising a trace metal, an ionic species, and/or a particle, and wherein the solvent comprises the alcohol or the ester.

48. The method of claim 36, further comprising preheating the azeotrope to a temperature at least about 20° C. below the boiling point before distilling the azeotrope in the first distillation column, wherein the preheating is performed by a preheater upstream of and in fluid communication with the first distillation column.

49. The method of claim 36, further comprising passing the azeotrope through a first filter unit upstream of the first distillation column, wherein the first filter unit comprises a first housing and at least one first filter in the first housing, and the at least one first filter comprises a filtration medium.

50. The method of claim 49, wherein the filtration medium in the at least one first filter comprises a polyolefin, a polyamide, a fluoropolymer, or a copolymer thereof.

51. The method of claim 50, wherein the filtration medium in the at least one first filter comprises polypropylene or polytetrafluoroethylene.

52. The method of claim 49, wherein the filtration medium in the at least one first filter has an average pore size from about 50 nm to about 250 nm.

53. The method of claim 49, wherein the at least one first filter is a particle removal filter.

54. The method of claim 36, further comprising passing the finished goods grade solvent through a second filter unit downstream of the second distillation column and optionally through an optional third filter unit downstream of the second filter unit, wherein the second filter unit comprises a second housing and at least one second filter in the second housing, and the at least one second filter comprises a filtration medium, and wherein the third filter unit, if present, comprises a third housing and at least one third filter in the third housing, and the at least one third filter comprises a filtration medium.

55. The method of claim 54, wherein the filtration medium in the at least one second filter comprises a polyolefin, a polyamide, a fluoropolymer, or a copolymer thereof.

56. The method of claim 55, wherein the filtration medium in the at least one second filter comprises nylon, polyolefin, or polytetrafluoroethylene.

57. The method of claim 54, wherein the filtration medium in the at least one second filter has an average pore size from about 2 nm to about 10 nm.

58. The method of claim 54, wherein the at least one second filter and/or the at least one third filter, if present, is a particle removal filter.

59. The method of claim 54, further comprising recirculating the finished goods grade solvent exiting the second filter unit and the third filter unit, if present.

60. The method of claim 59, wherein the recirculating comprises moving the finished goods grade solvent exiting the second filter unit to a distilled solvent tank and subsequently passing the finished goods grade solvent through the second filter unit and the third filter unit, if present, and the distilled solvent tank is between and in fluid communication with the second distillation column and the second filter unit.

61. The method of claim 36, further comprising refluxing the intermediate grade solvent exiting the bottom of the second distillation column.

62. The method of claim 36, further comprising moving the finished goods grade solvent to a product container downstream of and in fluid communication with the second distillation column.

63. The method of claim 36, wherein the finished goods grade solvent comprises the ester at a concentration less than about 200 ppm.

64. The method of claim 36, wherein the finished goods grade solvent comprises the ester at a concentration less than about 150 ppm.

65. The method of claim 36, wherein the finished goods grade solvent comprises the ester at a concentration less than about 100 ppm.

66. The method of claim 36, wherein the finished goods grade solvent comprises less than about 0.05 ppb Al as trace metal.

67. The method of claim 36, wherein the finished goods grade solvent comprises less than about 0.05 ppb Mn as trace metal.

68. The method of claim 36, wherein the finished goods grade solvent comprises less than about 0.05 ppb Zn as trace metal.

69. The method of claim 36, wherein the finished goods grade solvent comprises less than about 0.05 ppb Fe as trace metal.

70. The method of claim 36, wherein the finished goods grade solvent comprises less than about 0.05 ppb Na as trace metal.

71. The method of claim 36, wherein the finished goods grade solvent comprises water at a concentration less than about 50 ppm.
